(19) United States
(12) Patent Application Publication
(10) Pub. No.: US 2023/0212584 A1
(43) Pub. Date: Jul. 6, 2023

(54) MEDICAMENT USED FOR TREATING CHOLANGIOCARCINOMA WITH KRAS MUTATIONS

(71) Applicants: GENOMICARE BIOTECHNOLOGY (SHANGHAI) CO., LTD., SHANGHAI (CN); GENOMICARE BIOTECHNOLOGY LA CO. LTD., SHANGHAI (CN); GENOMICARE CLINICAL LABORATORY, QIDONG, QIDONG (CN)

(72) Inventors: QIANG XU, SHANGHAI (CN); GUAN WANG, SHANGHAI (CN); JINWANG WEI, SHANGHAI (CN); KUN WANG, SHANGHAI (CN)

(21) Appl. No.: 17/740,792

(22) Filed: May 10, 2022

(30) Foreign Application Priority Data
Jan. 6, 2022 (CN) 202210010315.9

(51) Int. Cl. C12N 15/113; A61K 31/565; A61K 31/138; A61K 31/4535; A61K 31/40; A61K 31/085; A61K 31/352; A61K 31/437; A61K 31/436; A61K 31/381; A61P 35/00

(52) U.S. Cl. CPC C12N 15/1138; A61K 31/565; A61K 31/138; A61K 31/4535; A61K 31/40; A61K 31/085; A61K 31/352; A61K 31/437; A61K 31/436; A61K 31/381; A61P 35/00; C12N 2310/14

(57) ABSTRACT

The present disclosure relates to a medicament for treating cholangiocarcinoma with KRAS mutations. Specifically, the present invention relates to a method of ER inhibitors for the specific treatment of cholangiocarcinoma with KRAS mutations, and to a use of ER inhibitors in the preparation of a medicament for the treatment of cholangiocarcinoma with KRAS mutations. New therapeutic regimens for patients suffering from cholangiocarcinoma with KRAS mutations are provided to improve the possibility of therapeutic benefit for patients.

### CROSS REFERENCE TO RELATED APPLICATION

This application claims priority to Chinese Patent Application No. 202210010315.9, having a filing date of Jan. 6, 2022, which is incorporated herein by reference for all purposes.

### TECHNICAL FIELD

The present disclosure relates to a method for the treatment of cholangiocarcinoma with KRAS mutations by using ER inhibitors and to a use of ER inhibitors in the preparation of a medicament for the treatment of cholangiocarcinoma with KRAS mutations.

### BACKGROUND

Cholangiocarcinomas (CCA) include intrahepatic cholangiocarcinoma (iCCA), perhilar/hilar cholangiocarcinoma (pCCA), and extrahepatic cholangiocarcinoma (ECCA), and the incidence and mortality rates thereof continue to increase worldwide, mainly due to the increase in ICCA. Cholangiocarcinoma is a hepatobiliary malignancy second only to hepatocellular carcinoma, accounting for approximately 3% of digestive tumors.

Due to the lack of appropriate diagnostic biomarkers, the vast majority of patients with cholangiocarcinoma are in the middle and advanced stage of the disease at the time of diagnosis. Most patients (>65%) have unresectable tumor, and the few patients who undergo surgery have a high recurrence rate. The current standard treatment regimen is gemcitabine in combination with platinum compounds, with a median survival of less than 12 months and a 5-year survival rate of only 5-15%.

As the treatment regimens for cholangiocarcinoma are limited and the overall prognosis is poor, new treatment methods for cholangiocarcinoma may bring hope for cholangiocarcinoma patients. With the development of tumor genome research, targeted therapies have brought additional benefits to cholangiocarcinoma patients. In April 2020, Pemazyre® became the first medicament approved by FDA to target FGFR2 fusion for the treatment of cholangiocarcinoma.

The frequency of KRAS mutations in cholangiocarcinoma ranges from 9% to 40%. KRAS mutations are associated with neuroinfiltration of the tumor, late staging, and poor prognosis. KRAS is one of the most important tumor driver genes and is second only to P53 and IDH in the incidence of cholangiocarcinoma. Current research shows that surgical resection is clinically possible in only 15% of patients, and median survival is usually less than 3 years. For patients with advanced or unresectable tumor, local and systemic chemotherapy are the main treatment options; however, the efficacy is poor.

Although the frequency of KRAS mutations is known to be high, direct targeting of KRAS remains challenging. At present, only one drug targeting the KRAS G12C mutations has been approved for the treatment of non-small cell lung cancer, and no treatment regimen specifically targeting cholangiocarcinoma with KRAS mutations has been reported in the literature.

### BRIEF SUMMARY OF THE DISCLOSURE

By analyzing gene expression and prognostic data from cholangiocarcinoma patients, the present inventors found that the growth of cholangiocarcinoma with KRAS mutations can be inhibited specifically by inhibiting ERs, and thus inhibiting ERs may be a potential regimen for treating cholangiocarcinoma.

First, in a first aspect of the present disclosure, the present disclosure relates to a method for treating cholangiocarcinoma with KRAS mutations in a subject, comprising the step of inhibiting the expression of estrogen receptors (ERs) or down-regulating estrogen receptors.

In some embodiments, the step of said method is silencing, knocking out, or knocking down the ESR1 gene. Further, said silencing, knocking out, or knocking down is performed with siRNAs, a sgRNAs or vectors constructed with shRNAs.

In other embodiments, the step of said method is administering a therapeutically effective amount of an ER inhibitor compound to said subject.

In a preferred embodiment, said ER inhibitor is selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof; or a combination thereof.

In a further preferred embodiment, said ER inhibitor is selected from Fulvestrant or a pharmaceutically acceptable salt thereof. Optionally, said ER inhibitor is a combination of Fulvestrant with one or more-compounds selected from the group consisting of Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof.

In a preferred embodiment, said mutations in the KRAS gene include, but are not limited to, one or more selected from the group consisting of p.G12V, p.G12C, p.G12D, p.G12A, p.G12R, p.G12S, p.G12F, pG13A, pG13E, pG13C, pG13R, pG13D, pG13S, pG13V, p.V14I, p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N.

In another aspect, the present disclosure also relates to a kit for the treatment of cholangiocarcinoma with KRAS mutations, comprising a reagent for inhibiting the expression of estrogen receptors or down-regulating estrogen receptors.

In some embodiments, said reagent for inhibiting the expression of estrogen receptors or down-regulating estrogen receptors is preferably siRNAs, sgRNAs or vectors constructed with shRNA.

In a preferred embodiment, said siRNAs, sgRNAs or vectors constructed with shRNA target the ESR1 gene.

In another aspect, the present disclosure also relates to ER inhibitor compounds, which are used for treating cholangiocarcinoma with KRAS mutations in a subject.

In some embodiments, said ER inhibitor compounds are selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof; preferably Fulvestrant; or a combination thereof.

In another aspect, the present disclosure also relates to a reagent for silencing, knocking out, or knocking down the ESR1 gene, which is used for treating cholangiocarcinoma with KRAS mutations in a subject.

In some embodiments, said reagent is selected from siRNAs, sgRNAs, or vectors constructed with shRNAs targeting the ESR1 gene.

In some embodiments, said reagent is selected from protein degradations targeted chimeric (PROTAC) medicament targeting an ER protein.

In another aspect, the present disclosure also relates to a use of a substance having inhibitory effects on ERs in the preparation of a medicament for the treatment of cholangiocarcinoma with KRAS mutations.

In some embodiments, wherein said substance is a substance that inhibits the expression of estrogen receptors or down-regulates estrogen receptors. Said substance is a substance used for silencing, knocking out, or knocking down the ESR1 gene. Preferably, said substance is siRNAs, sgRNAs, or shRNAs targeting the ESR1 gene.

In some other embodiments, said substance is an ER inhibitor compound, preferably selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof more preferably Fulvestrant; or a combination thereof.

In another aspect, the present disclosure relates to a use of Fulvestrant in combination with a second therapeutic agent in the preparation of a medicament for the treatment of cholangiocarcinoma with KRAS mutations.

In some embodiments, wherein said second therapeutic agent is a siRNA, sgRNA, or a shRNA targeting the ESR1 gene or other ER inhibitor compounds. Said other ER inhibitor compounds are preferably selected from the group consisting of Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof or a combination thereof.

In a preferred embodiment, wherein said mutations in the KRAS gene include, but are not limited to, one or more selected from the group consisting of p.G12V, p.G12C, p.G12D, p.G12A, p.G12R, p.G12S, p.G12F, pG13A, pG13E, pG13C, pG13R, pG13D, pG13S, pG13V, p. V14I, p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N.

In another aspect, the present disclosure also relates to a method for determining whether an ER inhibitor can be used to contact cholangiocarcinoma tumor cells to inhibit the growth or proliferation of said tumor cells, said method comprises determining the KRAS gene in said tumor cells, wherein the presence of a mutation in the KRAS gene indicates that the growth or proliferation of said tumor cells can be inhibited by the ER inhibitors.

In a preferred embodiment, said mutations in the KRAS gene include, but are not limited to, one or more of: p.G12V, p.G12C, p.G12D, p.G12A, p.G12R, p.G12S, p.G12F, pG13A, pG13E, pG13C, pG13R, pG13D, pG13S, pG13V, p.V14I, p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N; wherein the presence of said mutation indicates that the growth or proliferation of said tumor cells can be inhibited by the ER inhibitors.

### BRIEF DESCRIPTION OF THE FIGURES

FIG. 1: Survival plots of high expression of ESR1 (selected patients) versus low expression of ESR1 (unselected patients) in patients with KRAS mutant-type cholangiocarcinoma.

FIG. 2: Survival plots of high expression of ESR1 (selected patients) versus low expression of ESR1 (unselected patients) in patients with KRAS wild-type cholangiocarcinoma.

FIG. 3: CRISPR results of the iCSDB database.

FIG. 4: Medicament trial results.

### DETAILED DESCRIPTION OF THE DISCLOSURE

Before explaining in detail the embodiments of the present disclosure, it should be understood that the present disclosure is not limited to the contents or embodiments below, and that the present disclosure may have other embodiments. Furthermore, the phrases and terms used herein are for descriptive purposes and shall not be construed as limitations. The following definitions and methods are provided to better guide those skilled in the art to implement the present disclosure. Unless otherwise indicated, scientific and technical terms used herein have the meanings commonly understood by those skilled in the art.

RAS proto-oncogenes were originally transformed genes cloned from rat sarcoma virus. Weinberg et al., in 1982 discovered that activated HRAS genes were present in human bladder cancer cells, raising concerns about the role of RAS oncogenes in the development of human tumors. There are three RAS gene families associated with human tumors—HRAS, KRAS and NRAS, which are localized on chromosomes 11, 12, and 1, respectively. Among them, KRAS has the greatest impact on human cancers, acting as a molecular switch: when normal, it controls the pathways that regulate cell growth; and when abnormal, for example there is a KRAS mutation, the gene is permanently activated, causing disruption of intracellular signaling, leading to continued cell growth and preventing apoptosis, and thus become cancerous. KRAS mutations are common in colorectal cancer, pancreatic cancer, non-small cell lung cancer, endometrial cancer, and biliary tract cancer and the like.

In the context of the present disclosure, a hormone receptor refers to a protein molecule that specifically binds to a hormone to form a hormone-receptor complex, thereby enabling the hormone to perform its biological function. The term “estrogen receptors (ERs)” includes two major groups: 1) classical nuclear receptors, including estrogen receptor α (ERα) and estrogen receptor β (ERβ), which are both located in the nucleus, mediating genotypic effects of estrogen, i.e., genotypic regulatory effects by regulating the transcription of specific target genes; and 2) membrane receptors, including the membrane components of the classical nuclear receptors, as well as GPER1 (GPR30), Gaq-ER, and ER-X, which belong to the G protein-coupled receptor family, which can mediate rapid non-genotypic effects and exert transcriptional regulation through the second messenger system. The role of ERs is to provide binding sites for estrogens that are important for reproductive development and reproductive function. ER binding to estrogens leads to gene expression and controls the activation of signaling pathways, thereby regulating the cell growth process and controlling the cell cycle. The ESR1 gene is located on human chromosome 6q25.1, is 472929 bp in full-length and encodes ERα (i.e., estrogen receptor α), which is closely related to immunity.

Typically, non-restricted examples of ER (estrogen receptor) inhibitors include but are not limited to, Fulvestrant, Tamoxifen, Bazedoxifene, Raloxifene, Toremifene, Lasofoxifene, Ospemifene, Elacestrant, Amcenestrant, Giredestrant, Rintodestrant, AZD9833, LY3484356, ZN-c5, ARV-471, OP-1250, ZB716, D-0502, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502 and the like.

The present disclosure firstly relates to a method for treating cholangiocarcinoma with KRAS mutations in a subject, comprising the step of inhibiting the expression of estrogen receptors (ER) or down-regulating estrogen receptors.

In the context of the present disclosure, the KRAS mutations refer to the occurrence of an addition, substitution, or deletion in the KRAS gene, thereby altering its normal function and activating the mutated KRAS protein.

In the context of the present disclosure, KRAS-activating mutation refers to the occurrence of an addition, substitution, or deletion in the KRAS gene, thereby altering its normal function, resulting in an increased ability of the mutant cells to grow, migrate, transform, invade, and cause cancer and the like.

As used herein, the terms “subject” and “patient” are used interchangeably to refer to mammals requiring treatment, such as pets (e.g., dog, cat, and the like), livestock (e.g., cattle, pig, horse, sheep, goat, and the like), and laboratory animals (e.g., rat, mice, guinea pigs and the like). Preferably, it is a human in need of treatment.

The term “treatment” as used in this application includes alleviating, mitigating or ameliorating a disease or condition, preventing other symptoms, suppressing a disease or condition, such as prophylactically and/or therapeutically stopping or delaying the development of a disease or condition, relieving symptoms, causing them to subside and the like. In some embodiments, the treatment comprises extending progression-free survival. In other embodiments, the treatment comprises reducing the relative risk of disease development compared to other treatments. In some embodiments, other treatment options include, but are not limited to, hormone therapy (e.g., anti-estrogen therapy, and the like).

In some embodiments, said treatment method described in the present disclosure comprises the step of silencing, knocking out, or knocking down the ESR1 gene. Among them, preferably, said silencing, knocking out, or knocking down is performed by RNA interference (RNAi) or CRISPR gene editing techniques; further, preferably, said silencing knocking out or knocking down is performed using siRNAs, sgRNAs, or vectors constructed with shRNAs targeting the ESR1 gene.

In other embodiments, said treatment method comprises the step of administering a therapeutically effective amount of ER inhibitor compounds to said subject.

In a preferred embodiment, wherein said ER inhibitor is selected from the group consist of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof; or a combination thereof. More preferably, said ER inhibitor is selected from Fulvestrant or a pharmaceutically acceptable salt thereof; optionally, Fulvestrant may also be administered in combination with other ER inhibitors, or a pharmaceutically acceptable salt thereof as described above.

The term “combine” or “combination” of medicament means a product resulting from a mixture or combination of more than one active ingredient; it includes both fixed and non-fixed combinations of active ingredients. A “fixed combination” is one in which the active ingredient (e.g., Fulvestrant and other active agents) is administered simultaneously to a patient as a single entity or dose. A “non-fixed combination” means that the active ingredient and the other active agent are administered simultaneously, separately, or sequentially to the patient as separate entities or doses with no specific time interval between them. Non-fixed combinations are also applicable to cocktail, such as the administration of three or more active ingredients.

In the context of the present disclosure, the term “application” or “administration” refers to providing an effective level of said active compound in the patient.

As used herein, a “therapeutically effective amount” is an amount sufficient to elicit a target biological response. As understood by those skilled in the art, the effective amount of a compound of the present disclosure may vary depending on factors such as the biological target, the pharmacokinetics of the compound, the disease being treated, the mode of administration, and the age health and symptoms of the subject. Unless specified otherwise, a “therapeutically effective amount” of a compound as used herein is an amount sufficient to provide therapeutic benefit in the treatment of a disease, disorder, or condition, or to delay or minimize one or more symptoms associated with a disease, disorder, or condition. The therapeutically effective amount of a compound is the amount of the therapeutic agent that, when used alone or in combination with other therapies, provides a therapeutic benefit in the treatment of the disease, disorder, or condition. The term “therapeutically effective amount” may include an amount that improves overall treatment, reduces, or avoids the symptoms or causes of a disease or condition, or enhances the therapeutic effect of other therapeutic agents.

The term “pharmaceutically acceptable salt” refers to a salt of a compound of the present disclosure which is suitable for contact with a patient's tissues within the scope of reliable medical judgment, producing no undue toxicity, irritation, metabolic reactions and the like. Salts may include pharmaceutically acceptable base addition salts generated by association with metals or amines, such as alkali metal and alkaline earth metal hydroxides or organic amines. Examples of metals used as cations are sodium, lithium, potassium, magnesium, calcium, and the like. Examples of amines are tetramethylammonium, tetraethylammonium, methylamine, dimethylamine, trimethylamine, ethylamine, triethylamine, N,N′-Dibenzylethylenediamine, chloroprocaine, choline, diethanolamine, ethylenediamine, N-methylglucamine, and procaine. They may also be prepared from inorganic acids as sulfates, pyrosulfates, bisulfates, sulfites, bisulfites, nitrates, phosphates, monohydrogen phosphates, dihydrogen phosphates, metaphosphates, pyrophosphates, chlorides, bromides, iodides and the like. Said salts include, but are not limited to, hydrobromide, hydrochloride, sulfate, bisulfate, nitrate, acetate, oxalate, valerate, oleate, palmitate, stearate, laurate, borate, benzoate, lactate, phosphate, toluene sulfonate, citrate, maleate, fumarate, succinate, tartrate, naphthoic acid, methanesulfonate, glucosinolate, lactate, lauryl sulfonate, and hydroxyethyl sulfonate. They may further be prepared by salts prepared from organic acids, such as aliphatic mono- and di-carboxylic acids, phenyl-substituted alkanoic acids, hydroxyalkanoic acids, alkanedioic acids, aromatic acids, aliphatic and aromatic sulfonic acids. Said salts include, but are not limited to, acetate, propionate, octanoate, isobutyrate, oxalate, malonate, succinate, octanedioate, sebacate, fumarate, maleate, mandelate, benzoate, chlorobenzoate, methyl benzoate, dinitrobenzoic acid salt, naphthoic acid salt, benzene sulfonate, toluenesulfonate, phenylacetate, citrate, lactate, maleate, tartrate, methanesulfonate, and the like. It can also be a salt of amino acid, such as arginine salt, gluconate, galacturonate, and the like.

The pharmaceutical compound of the present disclosure, or a pharmaceutically acceptable salt thereof, or other forms, is administered to a subject. The compounds described herein, or a pharmaceutically acceptable salt thereof, may be administered to a subject by using any suitable delivery method, including surface, transintestinal, parenteral, transdermal, transmucosal, via inhalation, intracerebral pool, intradural, intravaginal, intravenous, intramuscular, subcutaneous, intradermal, and intravitreal administration.

The present disclosure also relates to a kit for the treatment of cholangiocarcinoma with KRAS mutations, comprising a reagent for inhibiting the expression of estrogen receptors or down-regulating estrogen receptors. Further, said reagent is a reagent for silencing, knocking out, or knocking down genes, preferably, said reagent is selected from siRNAs, sgRNAs, or vectors constructed with shRNAs.

On the other hand, the present disclosure also relates to an ER inhibitor compound which is used for the treatment of cholangiocarcinoma with KRAS mutations in a subject. Said ER inhibitor compound is preferably selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof more preferably Fulvestrant; or a combination thereof.

Moreover, the present disclosure relates to a use of a substance having an inhibitory effect on ERs in the preparation of a medicament for the treatment of cholangiocarcinoma with KRAS mutations. Said substance is preferably a substance that inhibits the expression of estrogen receptors or down-regulates estrogen receptors.

Furthermore, said substance is a substance for silencing, knocking out, or knocking down the ESR1 gene. Preferably, the substance is siRNAs, sgRNAs, or shRNAs targeting the ESR1 gene.

In some embodiments, wherein said substance is ER inhibitor compounds, preferably selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof or a combination thereof.

In addition, the present disclosure relates to a use of Fulvestrant in the preparation of a medicament for the treatment of cholangiocarcinoma with KRAS mutations.

As used herein, Fulvestrant is a new type of estrogen receptor antagonist—estrogen receptor down-regulation type of anti-breast cancer medicament that has been approved by the FDA for the treatment of ER-positive advanced breast cancer. It can act directly on ERs and achieve tumor suppression by inhibiting ERα transcription of ESR1 mutation. Fulvestrant has a CAS number of 129453-61-8 and the following structure:

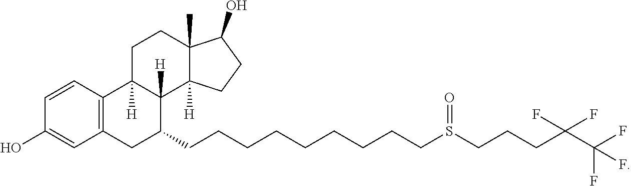

In some embodiments, the Fulvestrant is administered in combination with a second therapeutic agent; said second therapeutic agent is siRNA, sgRNA or shRNA targeting the ESR1 gene or other ER inhibitor compounds; said other ER inhibitor compounds are preferably selected from the group consisting of Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, or a pharmaceutically acceptable salt thereof; or a combination thereof.

In preferred embodiments, wherein said KRAS mutations comprise one or more selected from the group consisting of p.G12V, p.G12C, p.G12D, p.G12A, p.G12R, p.G12S, p.G12F, pG13A, pG13E, pG13C, pG13R, pG13D, pG13S, pG13V, p.V14I p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N.

In another aspect, the present disclosure also relates to a method for determining whether an ER inhibitor can be used to contact cholangiocarcinoma tumor cells to inhibit the growth or proliferation of said tumor cells, said method comprises determining the KRAS gene in said tumor cells, wherein the presence of a mutation in the KRAS gene indicates that the growth or proliferation of said tumor cells can be inhibited by the ER inhibitors.

In a preferred embodiment, said KRAS gene mutations include, but are not limited to, one or more of: p.G12V, p.G12C, p.G12D, p.G12A, p.G12R, p.G12S, p.G12F, pG13A, pG13E, pG13C, pG13R, pG13D, pG13S, pG13V, p. V14I, p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N; wherein the presence of said mutation indicates that the growth or proliferation of said tumor cells can be inhibited by the ER inhibitors.

Exemplary methods for detecting the presence of KRAS mutations in a biological sample comprise obtaining a biological sample (e.g., tumor-associated tissue or body fluid) from a test individual and contacting said biological sample with a compound or reagent capable of detecting KRAS-associated polypeptides or nucleic acids (e.g., mRNAs, genomic DNAs, or cDNAs). The assays of the present disclosure can be used to detect, for example, mRNAs, proteins, cDNAs or genomic DNAs in biological samples in vitro and in vivo. Detection techniques for mRNAs in vitro include Northern hybridization and in situ hybridization. Detection techniques for biomarker proteins in vitro include enzyme-linked immunosorbent assays (ELISA), protein blotting, immunoprecipitation, and immunofluorescence. Detection techniques for genomic DNA in vitro include Southern hybridization. Detection techniques for mRNAs in vivo include polymerase chain reaction (PCR), Northern hybridization and in situ hybridization.

The term “biological samples” is intended to include tissues, cells, biological fluids, and their isolates isolated from individuals, as well as tissues, cells, and fluids present within individuals.

Beneficial Effects of the Present Disclosure:

The present disclosure identifies the association of KRAS mutations with ESR1 gene in patients with cholangiocarcinoma through the gene mutation data, gene expression data, and survival data in the database, and confirms the association of KRAS mutation with estrogen pathway activation.

Furthermore, through the iCSDB database, the present disclosure queried that the growth of cholangiocarcinoma cell lines with KRAS mutations was more significantly inhibited after knocking down the ESR1 gene compared to KRAS wild type.

The present disclosure further verifies that the typical ER inhibitor (Fulvestrant) can significantly inhibit the cholangiocarcinoma cell line with KRAS mutations through cell viability experiments. It indicates that Fulvestrant and numerous ER inhibitors or inhibition methods may become a new approach for treating patients with cholangiocarcinoma.

### EXAMPLES

### Example 1: Determining the Association of KRAS Mutations with ESR1 Gene from the Patient Database

Relevant data (including patient's whole exon mutation data, transcriptome data, survival data, and the like) of cholangiocarcinoma patients from the GenomiCare Database (from GENOMICARE CLINICAL LABORATORY, QIDONG) were obtained.

The expression of each gene in the mutation positive group and the wild-type group of KRAS were compared and analyzed by AI neural network algorithm, and a group of genes with Causal Signal was obtained in the KRAS-mutant-type group. Functional clustering analysis of this group of genes was performed to obtain the gene signaling pathways with Causal Signal.

Table 1 below shows the results of the signaling pathway clustering analysis, which is an online analysis result of the commonly used analysis method Gene Set Enrichment Analysis (GSEA). After entering a group of genes with Causal Signal, the software will analyze, obtain a list, and rank the signal pathways most affected by KRAS mutations and according to the p value from minimum to maximum, then obtain the corresponding results—that estrogen-related pathways have the strongest causal relationship with positive KRAS mutations (i.e., KRAS mutations cause the activation of estrogen pathway).

TABLE 1

| | | | | | Error detec- |
|---|---|---|---|---|---|
| Name of gene sets | | Overlapping | | | tion rate |
| (The number of | Description of | gene numbers | | | (FDR) |
| genes (K)) | gene sets | (K) | k/K | p-value | q-value |
| Marker gene sets- | Genes defining | 21 | 0.105 | 9.82e⁻¹³ | 4.91e⁻¹¹ |
| Estrogen | the late response | | | | |
| responsive genes- | to estrogen | | | | |
| late stage [200] | | | | | |
| Marker gene sets- | Genes defining | 19 | 0.095 | 6.86e⁻¹¹ | 1.71e⁻⁹ |
| Estrogen | the early response | | | | |
| responsive genes- | to estrogen | | | | |
| early stage [200] | | | | | |
| Marker gene sets- | Genes encoding | 17 | 0.085 | 3.8e⁻⁹ | 6.33e⁻⁸ |
| E2F-targeting | cell cycle-related | | | | |
| genes [200] | targets of E2F | | | | |
| | transcription | | | | |
| | factors | | | | |
| Marker gene sets- | Genes | 16 | 0.08 | 2.58e⁻⁸ | 3.22e⁻⁷ |
| KRAS signaling | downregulated by | | | | |
| down-regulation | KRAS activation | | | | |
| genes [200] | | | | | |
| Marker gene sets- | Genes | 15 | 0.075 | 1.63e⁻⁷ | 1.63e⁻⁶ |
| KRAS signaling | upregulated by | | | | |
| up-regulation genes | KRAS activation | | | | |
| [200] | | | | | |
| Marker gene sets- | Genes encoding | 10 | 0.072 | 2.53e⁻⁵ | 2.11e⁻⁴ |
| coagulation genes | components of | | | | |
| [138] | the coagulation | | | | |
| | system; which are | | | | |
| | also upregulated | | | | |
| | in platelets | | | | |
| Marker gene sets- | Genes encoding | 10 | 0.05 | 5.36e⁻⁴ | 3.83e⁻³ |
| glycolysis [200] | proteins involved | | | | |
| | in glycolysis and | | | | |
| | gluconeogenesis | | | | |
| Marker gene sets- | Genes involved | 9 | 0.045 | 2.08e⁻³ | 1.15e⁻² |
| G2M checkpoints | in G2/M | | | | |
| [200] | checkpoints | | | | |
| Marker gene sets- | Genes defining | 9 | 0.045 | 3.24e⁻³ | 1.15e⁻² |
| Inflammatory | the inflammatory | | | | |
| response [200] | response | | | | |

131 cholangiocarcinoma patients were included in GenomiCare database in total, and the distribution of KRAS mutations tended to be more enriched in women (Table 2). Using the medical data of patients in GenomiCare database, the survival data of the corresponding patients could be compared by the high and low expression of ESR1.

TABLE 2
Clinical characteristics of 131 cholangiocarcinoma patients.

| | KRAS | | |
|---|---|---|---|
| | mutant-type | KRAS wild- | |
| Factors | group | type group | p |
| Gender | | | 0.057 |
| Female | 12 | 38 | |
| Male | 8 | 72 | |
| na | 0 | 1 | |
| Age (year) | | | 0.867 |
| >60 | 8 | 52 | |
| <60 | 11 | 58 | |
| na | 1 | 1 | |
| ESR1 genetic level | | | 0.303 |
| High (>median) | 7 | 56 | |
| Low (<median) | 13 | 55 | |

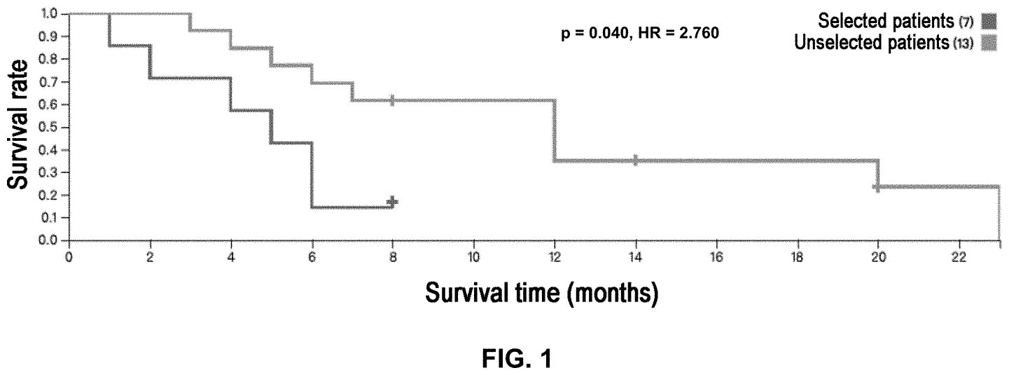

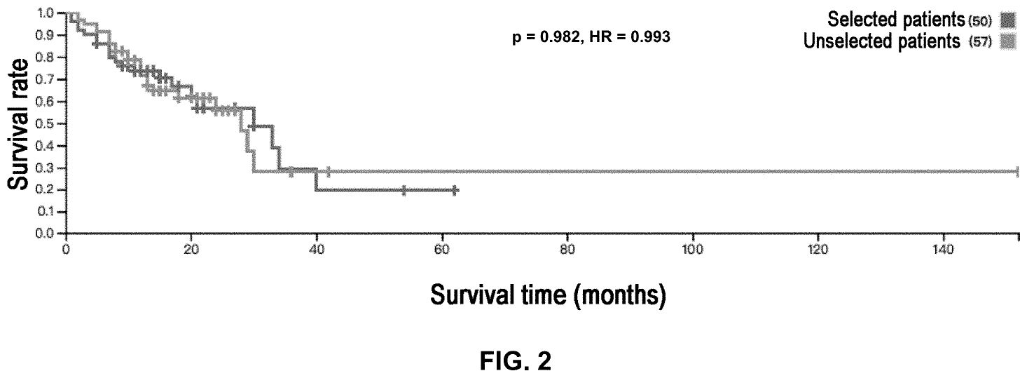

In cholangiocarcinoma patients with KRAS mutations, the median ESR1 expression in all patients was used as the cut-off value, above which was considered as a high expression and below which was considered as a low expression. The results showed that patients with a high expression of ESR1 (a key gene in the estrogen pathway) had a significantly worse survival rate than those with a low expression of ESR1 (FIG. 1), p=0.040, HR=2.760. However, in the wild-type group, there was no association between a high or low expression of ESR1 and the survival (FIG. 2), p=0.982, HR=0.993. It can be seen that ESR1 expression is significantly associated with prognosis in cholangiocarcinoma patients with KRAS mutations.

Further, after removing 3 patients without genders/ages, a COX multifactorial survival analysis was performed on the remaining 128 patients (Table 3).

TABLE 3
Multifactorial analysis of survival of cholangiocarcinoma patients.

| | | | KRAS | | KRAS | |
|---|---|---|---|---|---|---|
| | | | mutant-type | | wild-type | |
| | Total(N = 128) | | group(n = 19) | | group(n = 109) | |
| | HR | | HR | | HR | |
| Parameters | (95% Cl) | p | (95% Cl) | p | (95% Cl) | p |
| Genetic | 1.15 | 0.62 | 3.71 | 0.05 | 0.88 | 0.68 |
| level | (0.66-1.99) | | (1-13.79) | | (0.49-1.6) | |
| Gender | 1.29 | 0.37 | 1.78 | 0.33 | 1.09 | 0.79 |
| | (0.74-2.25) | | (0.55-5.74) | | (0.58-2.06) | |
| Age | 1.66 | 0.06 | 1.62 | 0.38 | 1.7 | 0.08 |
| | (0.99-2.78) | | (0.55-4.84) | | (0.93-3.11) | |
| KRAS | 4.40 | <0.001 | — | — | — | — |
| | (2.24-8.67) | | | | | |

At the overall level, gene expression, KRAS mutation status, gender, and age were included for multivariate analysis. Results indicated there was a significant difference in prognosis only when KRAS mutations were grouped (P<0.001), and age had a tendency to affect the prognosis.

After grouping patients according to KRAS, gene expression, gender, and age were included for multivariate analysis. Only in KRAS mutation group, the relationship between gene expression and prognosis was statistically different (P=0.05). In KRAS wild group, gene expression was not correlated with prognosis.

### Example 2: CRISPR Data Showed that KRAS Mutant Cholangiocarcinoma Cell Lines were More Sensitive to ER Knocking Out

iCSDB is an integrated database of human cell line CRISPR-Cas9 screening experiments. The two major sources of CRISPR-Cas9 screens are the DepMap portal and BioGRIDORCS. iCSDB contains 1375 genome-wide screens from 976 human cell lines, covering 28 tissues and 70 cancer types. Importantly, the database removes batch effects from different CRISPR libraries and converts screening scores to a single metric to estimate knocking out efficiency.

Knocking out a gene using CRISPR-Cas9 should have a similar effect with inhibiting the expression of that gene using a medicament. Thus, the use of CRISPR data can corroborate the effect of a specific gene on the growth of the corresponding cell.

Therefore, to further validate the preliminary results in Example 1, the effect of knocking out ESR1 (the gene encoding ER) in KRAS mutant-type and wild-type on growth in the database was observed to judge whether inhibition of ESR1 is a specific target of KRAS mutant-type cholangiocarcinoma.

After downloading the CRISPR-Cas9 results of all cell lines through the iCSDB website, all cholangiocarcinoma cell lines were screened and could be divided into KRAS-mutant-type group and KRAS wild-type group according to KRAS mutation status, with CRISPR score as the Y axis (Violin Plot) to obtain the correlation data between KRAS mutant-type group and wild-type group after knocking out of ESR1.

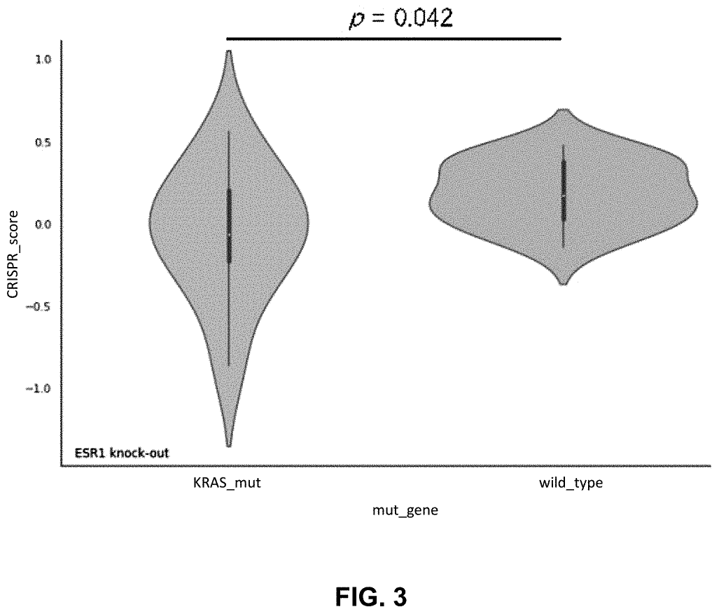

RESULTS: As shown in FIG. 3, relative to KRAS wild-type cholangiocarcinoma cell lines, knocking out of ESR1 significantly inhibited the growth of KRAS mutant-type cell lines (p=0.042). This predicts that inhibition of ERs can specifically inhibit the growth of KRAS mutant-type cholangiocarcinoma cell lines. It is hypothesized that by inhibiting ERs, silencing, knocking out, or knocking down ESR1 may be able to inhibit the growth of cholangiocarcinoma cells as described above, and thus it may have the ability to treat cholangiocarcinoma with KRAS mutations as described.

### Example 3: Cell Viability Assay, CellTiter-Glo (CTG) Method to Determine the Pharmacodynamic Activity of ER Inhibitor Compounds

To further validate the results in Example 2, a typical ER receptor antagonist, Fulvestrant, was used for further cell viability assays.

Fulvestrant was assayed for its anti-cholangiocarcinoma activity using the CTG method. The cell lines used were KRAS-mutated cholangiocarcinoma cell lines HUCCT1 and OZ, as well as KRAS wild-type cholangiocarcinoma cell line HCCC-9810. The efficacy of treatment by a medicament for cholangiocarcinoma was determined by comparing the difference in growth between KRAS-mutant-type group and wild-type group after treatment by a medicament. The specific steps were as follows:

1) Different experimental cell lines were sub-cultured into 96-well plates. 100 μL of PBS was added around the 96-well plates as a blank, where the cholangiocarcinoma cell lines used were HUCCT, OZ and HCCC-9810. The cells in the 96-well plates were cultured overnight at 37° C., 5% CO₂ and 95% humidity.

2) The cell culture medium in the 96-well plate was discarded and 100 μL of phenol red-free RPMI1640 culture medium containing 5% Charcoal Stripped FBS was added.

3) The cells in the 96-well plate with the changed medium were cultured at 37° C., 5% CO₂, 95% humidity for another 48 hours.

4) The cell culture medium in the 96-well plates was discarded and 80 μL phenol red-free RPMI1640 culture medium containing 5% Charcoal Stripped FBS was added.

5) The compound to be tested was dissolved with the appropriate solvent to form a stock solution and to perform a gradient dilution to obtain a 10-fold working concentration solution.

6) 10 μL of Fulvestrant solution was added to each well of a 96-well plate that has been inoculated with cells, and three replicate wells was set up for each cell concentration. The concentrations of the compounds to be tested were 0 μM, 1 μM, 2.5 μM, 5 μM, 10 μM, 15 μM and 20 μM, respectively.

7) 1 nM Estradiol solution was added to each well of a 96-well plate that has been inoculated with cells, and three replicate wells was set up for each cell concentration.

8) The cells in 96-well plates with Fulvestrant added were cultured at 37° C., 5% CO₂ and 95% humidity for 168 hours.

9) On the 11th day of the culture, CellTiter-Glo® reagent and medicament-treated cell culture plates were placed at room temperature for 30 minutes to equilibrate. 50 μL CellTiter-Glo® reagent was added to each well. Cells were shaken for 2 minutes on a fixed-rail shaker to fully lyse. Cell culture was placed at room temperature for 10 minutes to equilibrate. Chemiluminescence values were read with EnVision®.

10) All statistical analyses were performed with GraphPad® Prism™ Software using one-way ANOVA followed by Dunnett's post hoc test.

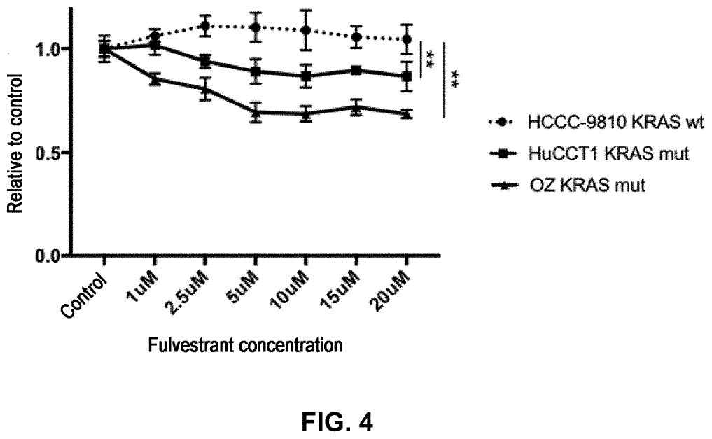

RESULTS: Fulvestrant significantly inhibited the growth of two KRAS mutant-type cholangiocarcinoma cell lines relative to KRAS wild-type cholangiocarcinoma cell lines (FIG. 4). 1 μM Fulvestrant already showed a significant growth inhibition of OZ, and with the increase of Fulvestrant concentration, the inhibition rate increased accordingly. HuCCT1 was less sensitive to Fulvestrant than OZ, and growth was significantly inhibited at the concentration higher than 2.5 μM, with a lower inhibition rate than OZ. The growth of two KRAS mutant-type cholangiocarcinoma cell lines was significantly inhibited compared to wild-type. This indicates that Fulvestrant may be used to inhibit KRAS mutation cholangiocarcinoma cells, and thus, may be used to treat the corresponding cholangiocarcinoma.

The preceding description of the disclosure is intended to be illustrative and not to limit the scope of the disclosure, and it should be understood by those skilled in the art that various changes in form and detail may be made thereto without departing from the claims and the scope of the disclosure. All published patents and patent applications cited herein are introduced herein for reference purposes. All other published references, documents, scientific literature and the like cited herein are incorporated herein by reference.

1. A method for treating cholangiocarcinoma with KRAS mutations in a subject, comprising the step of inhibiting the expression of estrogen receptors (ER) or down-regulating estrogen receptors.

2. The method of claim 1, wherein said step is silencing, knocking out, or knocking down the ESR1 gene with siRNAs, a sgRNAs or vectors constructed with shRNAs.

3. The method of claim 1, wherein said step is administering a therapeutically effective amount of an ER inhibitor compound to said subject.

4. The method of claim 3, wherein said ER inhibitor is selected from the group consisting of Fulvestrant, Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, and a pharmaceutically acceptable salt thereof.

5. The method of claim 3, wherein said ER inhibitor is selected from Fulvestrant or a pharmaceutically acceptable salt thereof.

6. The method of claim 3, wherein said ER inhibitor is a combination of Fulvestrant with one or more compounds selected from the group consisting of Tamoxifen, Raloxifene, Toremifene, Bardoxifene, Lasofoxifene, Ospemifene, Amcenestrant, AZD9833, Giredestrant, Elacestrant, LY3484356, ZN-c5, ARV-471, OP-1250, ZB716, D-0502, Rintodestrant, RU 58668, ZK 164015, ICI 182780, MPP dihydrochloride, PHTPP, AZD9496, Acolbifene, SAR439859, Nitromifene, Kaempferol, Brilanestrant, LSZ-102, H3B-5942, OP-1074, Endoxifen, GDC-0927 Racemate, GNE-502, and a pharmaceutically acceptable salt thereof.

7. A kit for the treatment of cholangiocarcinoma with KRAS mutations, comprising a reagent for inhibiting the expression of estrogen receptors or down-regulating estrogen receptors.

8. The kit of claim 7, wherein said reagent for inhibiting the expression of estrogen receptors or down-regulating estrogen receptors is a reagent for silencing, knocking out or knocking down genes.

9. The kit of claim 7, wherein said reagent for inhibiting the expression of estrogen receptors or down-regulating estrogen receptors is selected from siRNAs, sgRNAs or vectors constructed with shRNAs.

10. The kit of claim 9, wherein said siRNAs, sgRNAs or vectors constructed with shRNA target the ESR1 gene.

11. A method for determining that an ER inhibitor can be used to contact cholangiocarcinoma tumor cells to inhibit the growth or proliferation of said tumor cells, wherein said method comprises determining the KRAS gene in said tumor cells, wherein the presence of a mutation in the KRAS gene indicates that the growth or proliferation of said tumor cells can be inhibited by the ER inhibitor.

12. The method of claim 11, wherein said mutation in the KRAS gene is selected from one or more of: p.G12V, p.G12C, p.G12D, p.G12A, p.G12R, p.G12S, p.G12F, pG13A, pG13E, pG13C, pG13R, pG13D, pG13S, pG13V, p.V14I, p.L19F, p.Q22K, p.D33E, p.A59G, p.Q61E, p.Q61R, p.Q61H, p.Q61L, p.Q61P, p.Q61K, p.S65N, p.A146V, p.A146T, p.A146P, p.K117N; wherein the presence of said mutation indicates that the growth or proliferation of said tumor cells can be inhibited by the ER inhibitors.
